(19) United States
(12) Patent Application Publication
(10) Pub. No.: US 2008/0140180 A1
(43) Pub. Date: Jun. 12, 2008

(54) Vascular Position Locating Apparatus and Method

(75) Inventors: Mark Dolan, Santa Rosa, CA (US); David Simon, Boulder, CO (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/608,081

(22) Filed: Dec. 7, 2006

(51) Int. Cl. A61F 2/06; A61B 6/00

(52) U.S. Cl. 623/1.13; 600/431

(57) ABSTRACT

A branch vessel in a human patient is located using in vivo tracked field sensors where in one variation the sensor positions can be located by determining the positions of the sensors relative to a plurality of magnetic field sources of known location. This approach is used, for example, in locating the opening in a renal artery and positioning the proximal end of the AAA stent-graft adjacent to the opening. According to another embodiment, field sensors in combination with signal generators are placed in vivo to locate vasculature aspects. In a further embodiment, an in vivo sensor is positioned in a deployed prosthesis to create a reference for a prosthetic member having a sensor to track to during cannulation of the deployed prosthesis with the prosthetic member.

### FIELD OF THE INVENTION

The invention relates to prosthesis deployment and more particularly to locating a branch passageway in a human body such as a branch artery prior to prosthesis deployment or locating a passageway in a prosthesis prior to in vivo cannulation thereof.

### BACKGROUND OF THE INVENTION

Tubular prostheses such as stents, grafts, and stent-grafts (e.g., stents having an inner and/or outer covering comprising graft material and which may be referred to as covered stents) have been widely used in treating abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel. The abnormally dilated vessel has a wall that typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft spans across and extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened, and a graft is sutured into position bypassing the aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly to a portion where it spans across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter and self expands upon retraction or removal of the sheath at the target site. More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to distal end of the inner tube. Regarding proximal and distal positions referenced herein, the proximal end of a prosthesis (e.g., stent-graft) is the end closest to the heart (by way of blood flow) whereas the distal end is the end furthest away from the heart during deployment. In contrast, the distal end of a catheter is usually identified as the end that is farthest from the operator, while the proximal end of the catheter is the end nearest the operator. Once the catheter is positioned for deployment of the stent-graft at the target site, the inner tube is held stationary and the outer tube (sheath) withdrawn so that the stent-graft is gradually exposed and expands. An exemplary stent-graft delivery system is described in U.S. patent application Publication No. 2004/0093063, which published on May 13, 2004 to Wright et al. and is entitled Controlled Deployment Delivery System, the disclosure of which is hereby incorporated herein in its entirety by reference.

Although the endovascular approach is much less invasive, and usually requires less recovery time and involves less risk of complication as compared to open surgery, there can be concerns with alignment of asymmetric features of various prostheses in relatively complex applications such as one involving branch vessels. Branch vessel techniques have involved the delivery of a main device (e.g., a graft or stent-graft) and then a secondary device (e.g., a branch graft or branch stent-graft) through a fenestration or side opening in the main device and into a branch vessel.

The procedure becomes more complicated when more than one branch vessel is treated. One example is when an aortic abdominal aneurysm is to be treated and its proximal neck is diseased or damaged to the extent that it cannot support a reliable connection with a prosthesis. In this case, grafts or stent-grafts have been provided with fenestrations or openings formed in their side wall below a proximal portion thereof. The fenestrations or openings are to be aligned with the renal arteries and the proximal portion is secured to the aortic wall above the renal arteries.

To ensure alignment of the prostheses fenestrations and branch vessels, some current techniques involve placing guidewires through each fenestration and branch vessel (e.g., artery) prior to releasing the main device or prosthesis. This involves manipulation of multiple wires in the aorta at the same time, while the delivery system and stent-graft are still in the aorta. In addition, an angiographic catheter, which may have been used to provide detection of the branch vessels and preliminary prosthesis positioning, may still be in the aorta. Not only is there risk of entanglement of these components, the openings in an off the shelf prosthesis with preformed fenestrations may not properly align with the branch vessels due to differences in anatomy from one patient to another. Prostheses having preformed custom located fenestrations or openings based on a patient's CAT scans also are not free from risk. A custom designed prosthesis is constructed based on a surgeon's interpretation of the scan and still may not result in the desired anatomical fit. Further, relatively stiff catheters are used to deliver grafts and stent-grafts and these catheters can apply force to tortuous vessel walls to reshape the vessel (e.g., artery) in which they are introduced. When the vessel is reshaped, even a custom designed prosthesis may not properly align with the branch vessels.

U.S. Pat. No. 5,617,878 to Taheri discloses a method comprising interposition of a graft at or around the intersection of major arteries and thereafter, use of intravenous ultrasound or angiogram to visualize and measure the point on the graft where the arterial intersection occurs. A laser or cautery device is then interposed within the graft and used to create an opening in the graft wall at the point of the intersection. A stent is then interposed within the graft and through the created opening of the intersecting artery.

U.S. patent application Ser. No. 11/276,512 to Marilla, entitled Multiple Branch Tubular Prosthesis and Methods, filed Mar. 3, 2006, and co-owned by the assignee of the present application discloses positioning in an endovascular prosthesis an imaging catheter (intravenous ultrasound device (IVUS)) having a device to form an opening in the side wall of the prosthesis. The imaging catheter detects an area of the prosthesis that is adjacent to a branch passageway (e.g., a renal artery), which branches from the main passageway in which the prosthesis has been deployed. The imaging catheter opening forming device is manipulated or advanced to form an opening in that area of the prosthesis to provide access to the branch passageway. The imaging catheter also can include a guidewire that can be advanced through the opening.

Generally speaking, one challenge in prosthesis (e.g., stent graft) delivery/placement in the vicinity of one or more branch vessels is identifying and locating the position of branch vessels (e.g., arteries). Typically fluoroscopy is used to identify branch vessels. More specifically, fluoroscopy has been used to observe real-time X-ray images of the openings within cardiovascular structures such as the renal arteries during a stent-graft procedure. This approach requires one to administer a radiopaque substance, which generally is referred to as a contrast medium, agent or dye, into the patient so that it reaches the area to be visualized (e.g., the renal arteries). A catheter can be introduced through the femoral artery in the groin of the patient and endovascularly advanced to the vicinity of the renals. The fluoroscopic images of the transient contrast agent in the blood, which can be still images or real-time motion images, allow two dimensional visualization of the location of the renals.

The use of X-rays, however, requires that the potential risks from a procedure be carefully balanced with the benefits of the procedure to the patient. While physicians always try to use low dose rates during fluoroscopy, the duration of a procedure may be such that it results in a relatively high absorbed dose to the patient. Patients who cannot tolerate contrast enhanced imaging or physicians who must or wish to reduce radiation exposure need an alternative approach for defining the vessel configuration and branch vessel location.

Accordingly, there remains a need to develop and/or improve prosthesis deployment apparatus and methods for endoluminal or endovascular applications.

### SUMMARY OF THE INVENTION

The present invention involves improvements in prosthesis deployment apparatus and methods.

In one embodiment according to the invention, a method of locating a branch vessel in a human patient comprises tracking a sensor moving in a vessel in a first navigational direction (e.g., along a vessel wall); and detecting movement of the sensor in a direction generally orthogonal to the first navigational direction. The detected movement can be monitored to confirm if branch vessel detection occurred.

In another embodiment according to the invention, a method of positioning a tubular prosthesis in a passageway in a human body comprises advancing a tubular prosthesis through a vessel in a patient; obtaining the position in three dimensions of a portion of an opening to a branch vessel; and positioning the proximal end portion of the prosthesis at a predetermined distance from the branch vessel opening portion. In one example, the vessel can be the aorta of the patient and the branch vessel can be a renal artery.

In another embodiment according to the invention, a method of cannulating a bifurcated tubular prosthesis in vivo comprises positioning a bifurcated tubular prosthesis in the aorta of a patient having an ipsilateral leg and a truncated contralateral leg portion; positioning a first sensor in the truncated contralateral leg portion; obtaining the position in three dimensions of the first sensor; advancing a contralateral leg delivery catheter, which has a distal portion and a proximal portion and a second sensor coupled to the distal portion, toward the first sensor position; and monitoring the second sensor position in three dimensions relative to the first sensor position to guide the distal portion of the contralateral leg delivery catheter into the truncated contralateral leg portion.

In another embodiment according to the invention, a prosthesis delivery system comprises a stent-graft delivery catheter having a proximal end portion and a distal end portion; a first sensor coupled to the catheter distal end portion; a flexible member having a fixed end portion and a feeler end portion, the flexible member fixed end portion being secured to the catheter distal end portion; and a second signal sensor coupled to the flexible member feeler end portion and suspended thereby.

In another embodiment according to the invention, a prosthesis delivery system comprises a tubular prosthesis delivery sheath having a proximal end portion and a distal end portion; a tip member having a proximal end portion and a distal end portion, the tip member proximal end portion being releasably coupled to the sheath distal end portion; a first sensor coupled to the tip member; a flexible member having a fixed end portion and a feeler end portion, the flexible member fixed end portion being secured to the tip member; and a second sensor coupled to the flexible member and suspended thereby.

In another embodiment according to the invention, a stent-graft delivery system comprises a stent-graft delivery catheter having a proximal end portion and a distal end portion; a flexible member having a fixed end portion and a feeler end portion, the flexible member fixed end portion being secured to the catheter distal end portion; a first sensor coupled to one of the catheter distal end portion and the flexible member; a signal generator coupled to the other of the catheter distal end portion and the flexible member; and the one of the sensor and signal generator that is coupled to the flexible member being suspended thereby.

In another embodiment according to the invention, a stent-graft delivery system comprises a stent-graft delivery sheath having a proximal end portion and a distal end portion; a tip member having a proximal end portion and a distal end portion, the tip member being releasably coupled to the sheath distal end portion; a flexible member having a fixed end portion and a feeler end portion, the flexible member fixed end portion being secured to the tip member; a first sensor coupled to one of the tip member and the flexible member; a signal generator coupled to the other of the tip member and the flexible member; and the one of the sensor and signal generator that is coupled to the flexible member being suspended thereby and movable relative to the tip member.

According to another embodiment of the invention a probe for locating structure in a patient comprises an elongated member configured for endovascular delivery in a patient, the elongated member having a proximal end portion and a distal end portion; a first sensor coupled to the elongated member distal end portion; a flexible member having a proximal end portion and a distal end portion, the flexible member fixed end portion being secured to the elongated member distal end portion; and a second sensor coupled to the flexible member and suspended thereby.

Other features, advantages, and embodiments according to the invention will be apparent to those skilled in the art from the following description and accompanying drawings.

### BRIEF DESCRIPTION OF THE DRAWINGS

FIG. 1 diagrammatically illustrates one embodiment of a prosthesis delivery system in accordance with the invention.

FIG. 2 diagrammatically illustrates an electromagnetic field generating system for use with the prosthesis delivery system of FIG. 1.

FIG. 3 is a partial sectional view of a distal portion of the prosthesis delivery system of FIG. 1 coupled to the circuit of FIG. 2.

FIG. 4 schematically illustrates one embodiment of a multiple coil sensor which can be used in the various embodiments described herein.

FIG. 5 is an end view of the prosthesis delivery system of FIG. 1 taken from 5-5 in FIG. 1 showing optional sensors and accompanying carrier arms.

FIGS. 6-15 illustrate a method of stent-graft deployment in accordance with the invention, where FIGS. 6, 7, 8, and 9 illustrate advancing the prosthesis delivery system of FIG. 1 from a femoral artery to the vicinity of a renal artery; FIG. 10 depicts sensor movement into a renal artery indicating renal artery location; FIG. 11 depicts renal artery location confirmation; FIG. 12 depicts stent-graft deployment adjacent to the located renal artery; FIG. 13 depicts obtaining a position in three dimensions in the contralateral stent-graft short leg using a sensor; FIG. 14 illustrates cannulating the contralateral stent-graft short leg with a contralateral catheter having a sensor attached to a distal portion thereof; and FIG. 15 illustrates the full deployment of the modular bifurcated stent-graft of FIG. 14 with an optional distal bare spring wire.

FIGS. 16 and 17 are flow charts for the method of FIGS. 6-15.

FIG. 18 diagrammatically illustrates another embodiment of a prosthesis delivery system in accordance with the invention.

FIG. 19 provides a schematic sectional view to help illustrate a method of using the prosthesis delivery system of FIG. 18.

### DETAILED DESCRIPTION

The following description will be made with reference to the drawings where when referring to the various figures, it should be understood that like numerals or characters indicate like elements.

Regarding proximal and distal positions, the proximal end of the prosthesis (e.g., stent-graft) is the end closest to the heart (by way of blood flow) whereas the distal end is the end farthest away from the heart during deployment. In contrast, the distal end of the catheter is usually identified as the end that is farthest from the operator, while the proximal end of the catheter is the end nearest the operator. Therefore, the prosthesis (e.g., stent-graft) and delivery system proximal and distal descriptions may be consistent or opposite to one another depending on prosthesis (e.g., stent-graft) location in relation to the catheter delivery path.

Embodiments according to the invention facilitate mapping of one or more branch lumens in a patient prior to stent-graft deployment and/or locating a prosthesis lumen position prior to cannulation thereof. Branch lumens emanate from the intersection of a vessel (e.g., the aorta) and other attendant vessels (e.g., major arteries such as the renal, brachiocephalic, subclavian and carotid arteries). According to one embodiment of the invention, one or more sensors, which can be signal devices (e.g., magnetically sensitive, electrically conductive sensing coils, which can be referred to as antenna coils), are coupled to a prosthesis delivery catheter through a flexible member that allows the signal device(s) to move relative to the catheter.

In the case of magnetically sensitive, electrically conductive sensing coils, the coil positions can be located by determining the positions of the coils relative to a plurality of magnetic field sources of known location. Pre-specified electromagnetic fields are projected to the portion of the anatomical structure of interest (e.g., that portion that includes all prospective locations of the coils in a manner and sufficient to induce voltage signals in the coil(s). Electrical measurements of the voltage signals are made to compute the angular orientation and positional coordinates of the sensing coil(s) and hence the location of the vasculature and/or devices of interest. An example of sensing coils for determining the location of a catheter or endoscopic probe inserted into a selected body cavity of a patient undergoing surgery in response to prespecified electromagnetic fields is disclosed in U.S. Pat. No. 5,592,939 to Martinelli, the disclosure of which is hereby incorporated herein by reference in its entirety. Another example of methods and apparatus for locating the position in three dimensions of a sensor comprising a sensing coil by generating magnetic fields which are detected at the sensor is disclosed in U.S. Pat. No. 5,913,820 to Bladen, et al., the disclosure of which is hereby incorporated herein by reference in its entirety.

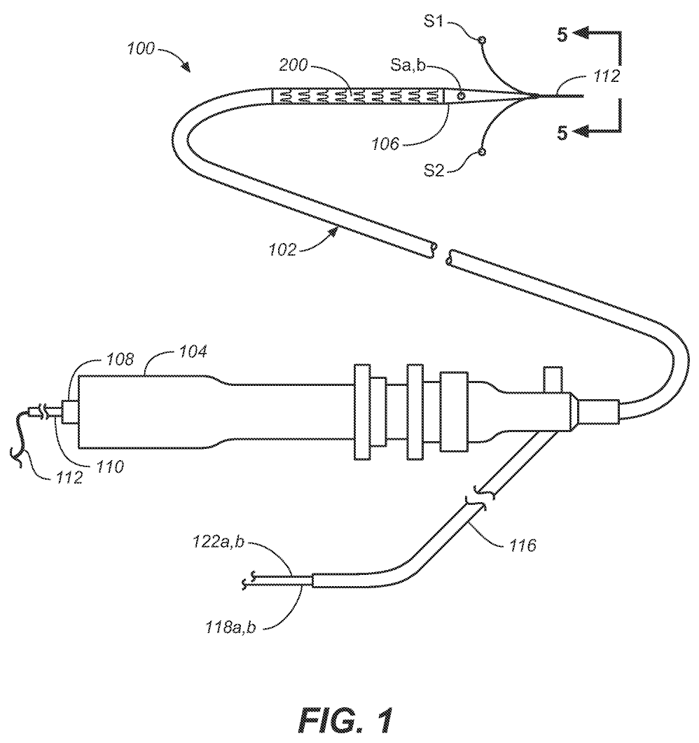

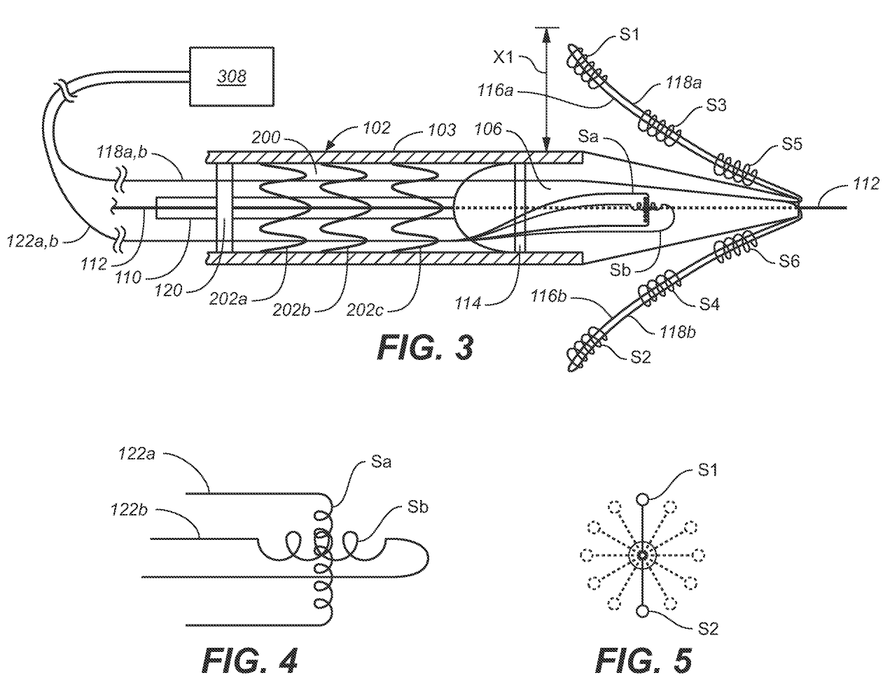

Referring to FIG. 1, a first embodiment of a prosthesis delivery system according to the invention is shown and generally designated with reference numeral 100. Prosthesis delivery system 100 comprises catheter 102, control handle 104, tapered tip member (or obturator) 106, which can form a portion of the distal end of the catheter. Handle 104 includes an inlet 108, through which central guidewire lumen 110 enters the handle and extends to flexible tapered tip 106, which has an axial bore for slidably receiving guidewire 112. Tapered tip member 106 is at the distal end of catheter sheath 103 (FIG. 3) and handle 104 is at to the proximal end of the catheter sheath. Guidewire 112 can be slidably disposed in guidewire lumen 110 and catheter 102 tracked thereover.

One or more sensors (S1, S2 . . . Sn) are suspended from tapered tip 106. Further, one or more sensors (Sa, Sb . . . Sn) are coupled to the tapered tip and can be secured to or embedded in the tapered tip as will be described in more detail below. Alternatively, sensors (Sa, Sb . . . Sn) can be coupled to the catheter sheath or guidewire lumen along the distal portion of the catheter sheath adjacent to the tapered tip.

When the prosthesis to be delivered is a self-expanding graft or stent-graft (such as stent-graft 200 shown in FIG. 3, it generally is radially compressed or folded and placed in the distal end portion of the delivery catheter and allowed to expand upon deployment from the catheter at the target site as will be described in detail below. Stent-graft 200 can include a plurality of undulating stent elements 202a,b,c to support the tubular graft material as is known in the art.

Referring to FIG. 3, catheter tube or sheath 103 (outer tube) and inner guidewire tube 110 are coaxial and arranged for relative axial movement therebetween. The prosthesis (e.g., stent-graft 200) is positioned within the distal end of outer tube 103 and in front of pusher member or stop 120, which is concentric with and secured to inner guidewire tube 110 and can have a disk or ring shaped configuration with a central access bore to provide access for guidewire tube 110. A radiopaque ring 114 can be provided on the proximal end of tapered tip 106 or the inside of sheath 103 to assist with imaging the tapered tip or distal end of sheath 103 using fluoroscopic techniques. Once the catheter is positioned for deployment of the prosthesis at the desired site, the inner member or guidewire lumen 110 with stop 120 are held stationary and the outer tube or sheath 103 withdrawn so that sheath 103 is displaced from tapered tip 106 and the stent-graft gradually exposed and allowed to expand. Stop 120 therefore is sized to engage the distal end of the stent-graft as the stent-graft is deployed. The proximal ends of the sheath 103 and inner tube or guidewire lumen 112 are coupled to and manipulated by handle 104. Tapered tip 106 optionally can include a stent graft proximal end holding mechanism to receive and hold the proximal end of the stent-graft so that the operator can allow expansion of the stent-graft proximal end during the last phase of its deployment. In this regard, any of the stent-graft deployment systems described in U.S. patent application Publication No. 2004/0093063, which published on May 13, 2004 to Wright et al. and is entitled Controlled Deployment Delivery System, the disclosure of which is hereby incorporated herein by reference in its entirety, can be incorporated into stent-graft delivery system 100.

In the embodiment shown in FIG. 3, a plurality of sensors are coupled to the catheter and suspended therefrom through flexible member 116a and a plurality of sensors are coupled to the catheter and suspended therefrom through flexible member 116b. Flexible members 116a,b, which can be wires, allow the sensors attached thereto to move toward or away from the catheter. Sensors S1, S3 and S5 are axially spaced from one another along flexible member 116a (with S1 at the feeler end of the flexible member) and electrically coupled to processor or measuring unit 308 through conductor or copper wire 118a, which can extend through the distal opening of tapered tip 106 and through guidewire lumen 110 before branching out to processor or measuring unit 308 in the vicinity of handle 104. Similarly, sensors S2, S4 and S6 are axially spaced from one another along flexible member 116a (with S2 at the feeler end of the flexible member) and electrically coupled to processor or measuring unit 308 through conductor or copper wire 118b, which can extend through the distal opening of tapered tip 106 and through guidewire lumen 110 before branching out to processor or measuring unit in the vicinity of handle 104. Each conductor or copper wire can be wound around a respective flexible member to secure the conductor and hence the sensors thereto. Each flexible member has a fixed end and a feeler end and each fixed end is attached to the distal end of tapered tip 106. In this manner, the flexible members can be used as feeler wires to find and position branch vessels such as the renal arteries.

Although the flexible members are each shown with three sensors, the number of sensors can vary. For example, a single sensor can be provided at each flexible member feeler end. However, three sensors suspended along a respective flexible member as shown in FIG. 3, provides a sufficient number of data points to provide a virtual image of the flexible member and, thus, provide a virtual image of the contour, orientation and/or direction of the branch vessel to determine, for example, if a branch vessel extends about 90 degrees or about 30 degrees from the vessel from which it branches.

In the illustrative embodiment of FIG. 4, a pair of sensors Sa and Sb are secured to the tapered tip to provide a reference signal. They can be embedded in or otherwise attached to tapered tip 106 and extended through guidewire lumen 110 and then coupled to processor or measuring unit 308. In an alternative embodiment, sensors Sa and Sb can be secured to a distal portion of catheter sheath 103 or guidewire lumen 110. Further and as shown in the embodiment illustrated in FIG. 4, sensors Sa and Sb can be coils that are oriented perpendicular to one to another. Similar perpendicular sensor pairs can be used in place of one or more of sensors S1-S6 shown in FIG. 3.

Referring to FIG. 5, where optional flexible members are shown in dashed line, it is to be understood that the number of flexible members having one or more sensors coupled or secured thereto or suspended thereby can vary. Further, a single flexible member with one or more sensors coupled or secured thereto can be used.

Each flexible member 116a and 116b can be made from shape memory material and provided with a preshaped memory set configuration such as the configuration shown in FIG. 3. For example, flexible members 116a and 116b can be nitinol wire and can be placed in the desired shape (e.g., that shown in FIG. 3) and heated for about 5-15 minutes in a hot salt bath or sand having a temperature of about 480-515° C. They can then be air cooled or placed in an oil bath or water quenched depending on the desired properties. In one alternative, flexible members 116a and 116b can be stainless steel and preshaped with known techniques to assume the configuration shown in FIG. 3.

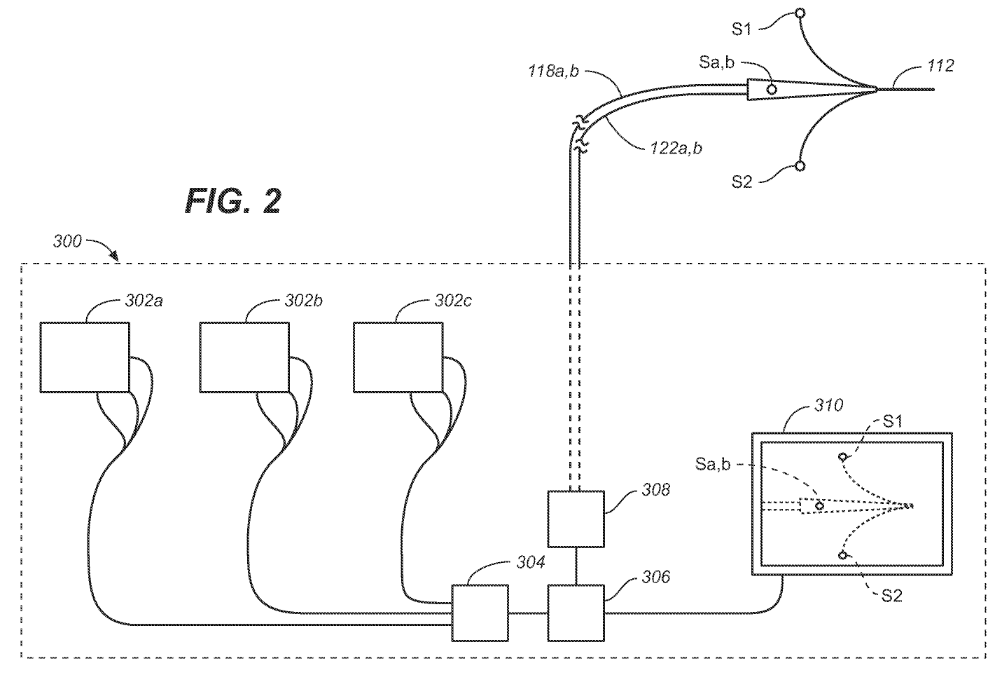

Any suitable electromagnetic field generating and signal processing circuit for locating sensor position in three dimensions can be used (see e.g., U.S. Pat. No. 5,913,820 to Bladen, et al. (supra) regarding magnetically sensitive, electrically conductive sensing coils (e.g., antenna coils)). Referring to FIG. 2, one such field generating and signal processing circuit configuration for generating magnetic fields at the location of the sensors and processing the voltage signals that the sensors generate in response to the generated magnetic fields, when the sensors are conductive sensing coils, is generally designated with reference numeral 300.

Circuit 300 generally includes three electromagnetic field (EMF) generators 302a, 302b, and 302c, amplifier 304, controller 306, measurement unit 308, and display device 310. Each field generator comprises three electrically separate coils of wire (generating coils) wound about a cuboid wooden former. The nine generating coils are separately electrically connected to amplifier 304, which is able, under the direction of controller 306, to drive each coil individually.

In use, controller 306 directs amplifier 304 to drive each of the nine generating coils sequentially. Once the quasi-static field from a particular generating coil is established, the value of the voltage induced in each sensing coil (S1-S6) by this field is measured by the measurement unit 308, processed and passed to controller 306, which stores the value and then instructs the amplifier 304 to stop driving the present generating coil and to start driving the next generating coil. When all generating coils have been driven, or energized, and the corresponding nine voltages induced into each sensing coil have been measured and stored, controller 306 calculates the location and orientation of each sensor relative to the field generators and displays this on a display device 310. This calculation can be carried out while the subsequent set of nine measurements are being taken. Thus, by sequentially driving each of the nine generating coils, arranged in three groups of three mutually orthogonal coils, the location and orientation of each sensing coil can be determined.

The sensor and generating coil specifications, as well as the processing steps are within the skill of one of ordinary skill of the art. An example of coil specifications and general processing steps that can be used are disclosed in U.S. Pat. No. 5,913,820 to Bladen, et al., the disclosure of which is hereby incorporated herein by reference in its entirety.

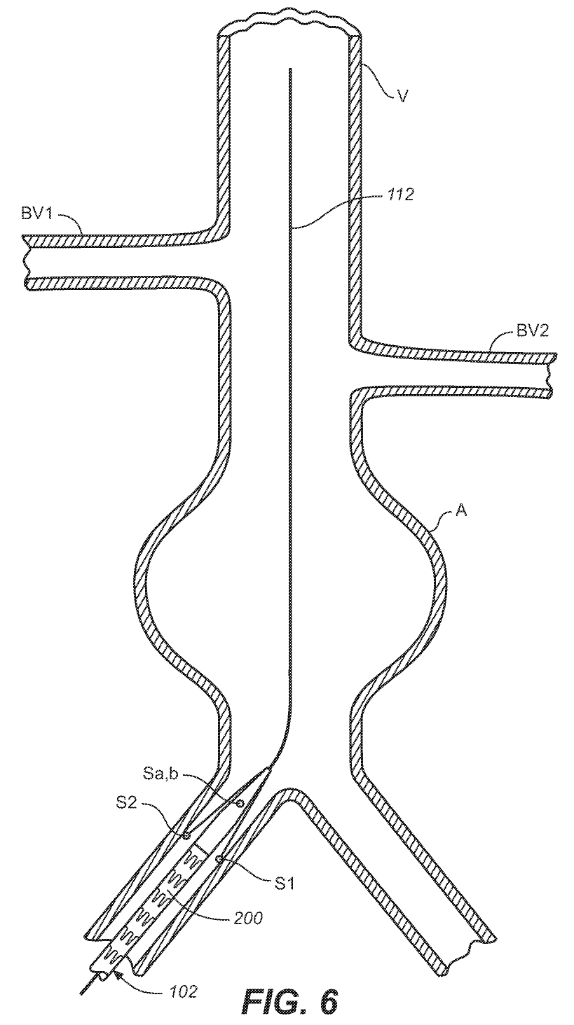

Referring to FIGS. 6-15, an exemplary operation of the system will now be described. For the purposes of the example, the procedure involves the endovascular delivery and deployment of an AAA bifurcated stent-graft.

Prior to the surgical procedure, the patient is scanned using either a CT or MRI scanner to generate a three-dimensional model of the vasculature to be tracked. Therefore, the aorta and branch vessels of interest (e.g., renal arteries) can be scanned and images taken therealong to create a three-dimensional pre-procedural data set for that vasculature and create a virtual model upon which real-time data will be overlayed. This information is stored in the system and is identified and accessible as a historical baseline image. Any portion of the aorta or branch vessels can be provided with fiducial markers (anatomic markers which are considered to provide a reliable reference to a particular body location) that are visible on the pre-procedural images and accurately detectable during the procedure as is known in the art.

The three magnetic field generators are positioned on the operating table to facilitate triangulation of the exact position of each sensor in three-dimensional space using xyz coordinates.

The patient is prepared for surgery and a cut is made down to a femoral artery and a guidewire (by itself or together with a guide catheter) inserted. A contrast agent catheter is delivered through the femoral artery and the vasculature perfused with contrast and a fluoroscopic image including the renal arteries taken. Using the fiducial markers, the processor orients or registers the previously acquired and stored three-dimensional image to the currently presented fluoroscopic X-ray image.

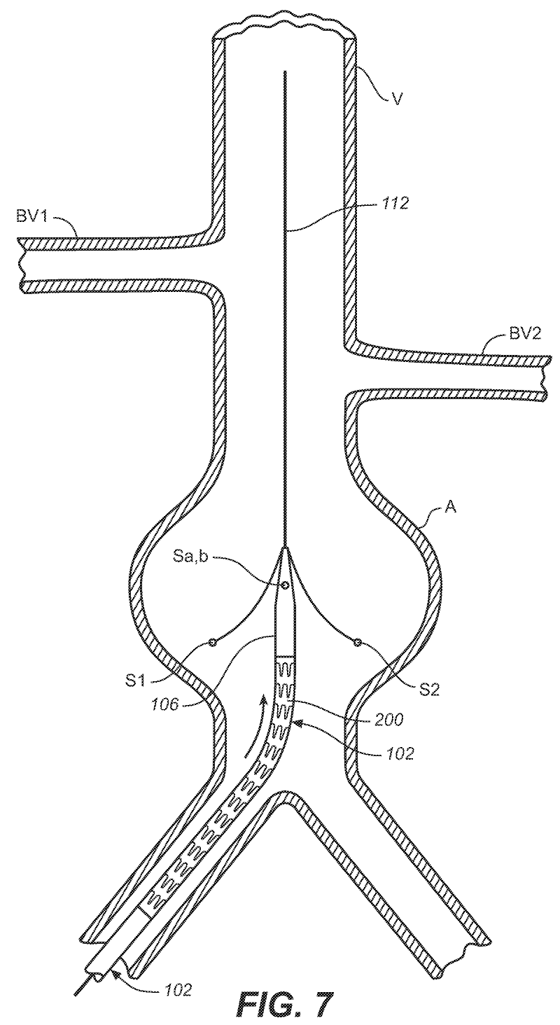

Referring to FIG. 6, the operator tracks catheter 102 over guidewire 112 toward aneurysm A and branch vessels BV1 and BV2, which branch from vessel V, which in this example is the aorta. The position of the distal end of the catheter is monitored virtually based on the known catheter dimensions entered into the processor and the signals from sensors (or coils) S1, S2, Sa and Sb, which identify their position in the three-dimensional model. The display will show the position of the sensors, which may be referred to as markers, tracking the profile of the vessel wall. The operator may visualize the displacement of sensors S1 and S2 as the tapered tip passes through aneurysm A (FIG. 7), where the walls of the aneurysm bulge so much that they do not contact or constrain flexible members 116a,b. The flexible members or feeler wires 116a,b are then free to move toward or to their undeformed free state (memory set configuration). In this state, end sensors S1 or S2 can be radially spaced a distance X1 (FIG. 3) measured from the juncture of the catheter and tapered tip in an orthogonal direction extending radially outward therefrom. X1 typically is about 18 mm to 36 mm, but can vary according to the application.

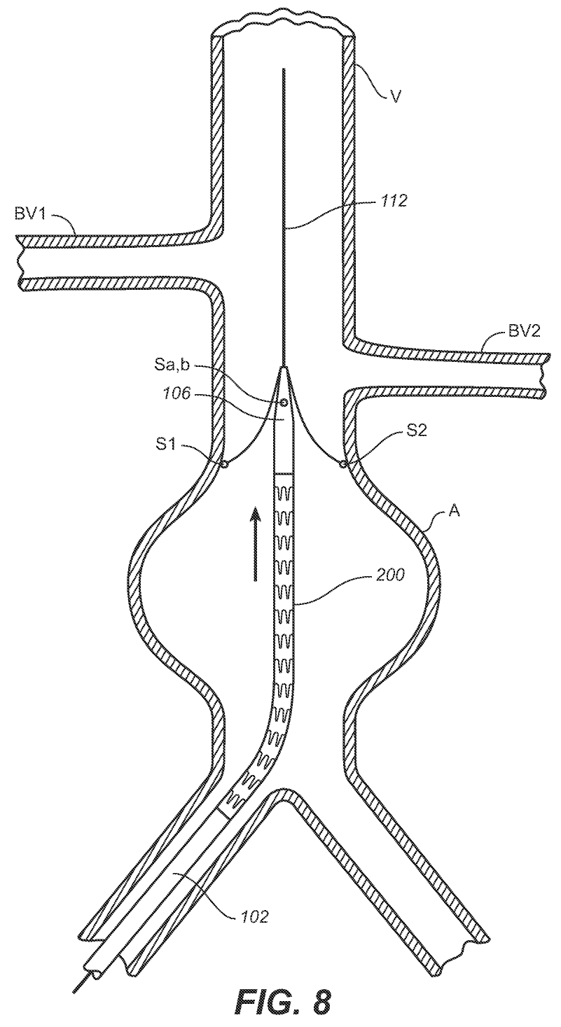

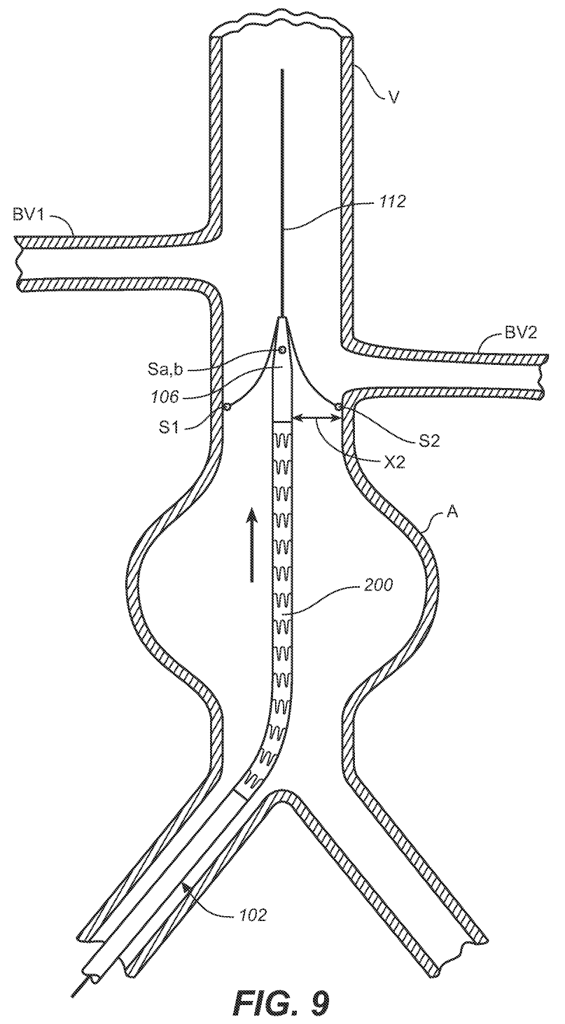

The catheter is further advanced and the sensors reach the aneurysm's proximal neck as shown in FIG. 8 where they move radially inward toward catheter tapered tip 106. Their position continues to be relayed to the operator as they move along the proximal neck to a point where they are radially spaced from the catheter a distance X2 (measured from the juncture of the catheter and tapered tip and in an orthogonal direction extending radially outward therefrom) as shown in FIG. 9.

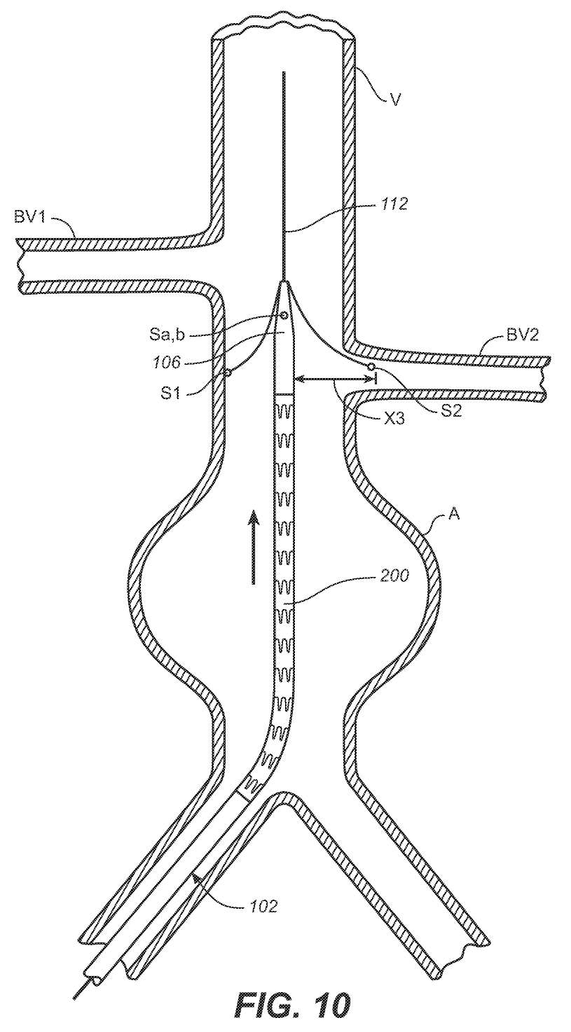

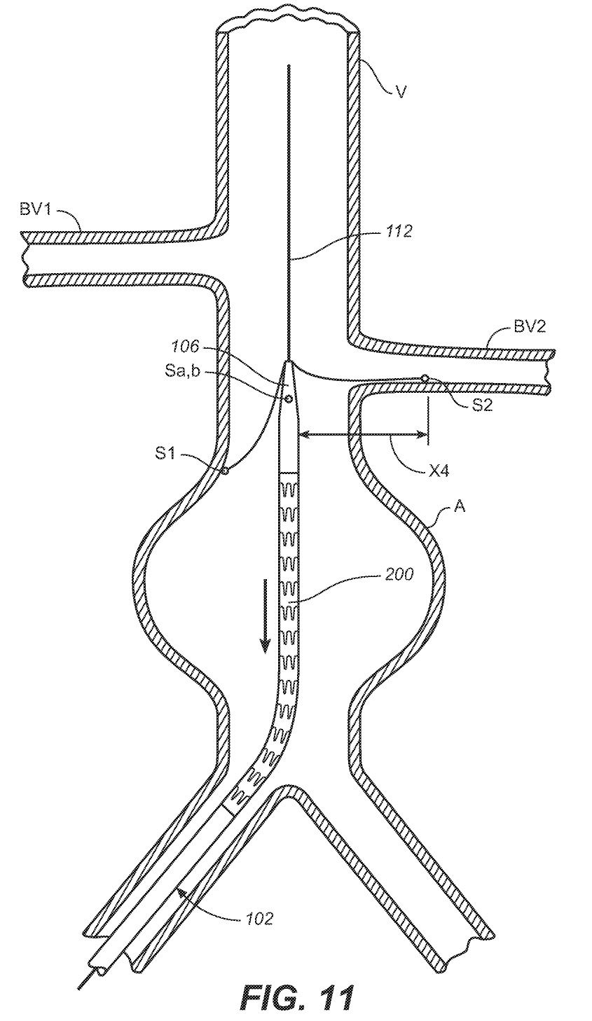

In the vicinity of the target location (e.g., the lower renal artery), which the operator can estimate based on the three-dimensional model and the sensor positions, the operator rotates and further advances the catheter to find the lower renal artery, which in this example corresponds to BV2. When a sensor indicates movement in a direction radial outward from tapered tip 106 that exceeds the expected position of the vessel wall, the operator can conclude that the renal artery has been found. Referring to FIG. 10, the position of the sensor can be determined and the determined position used to calculate the distance (e.g., distance X3) the sensor and the catheter measured from the juncture of the catheter and tapered tip in an orthogonal direction extending radially outward therefrom as an indicator of the sensor being located in the renal artery. Alternatively, the operator can simply qualitatively track the magnitude of sensor radial outward movement on the three-dimensional model as displayed on the monitor as an indicator of the renal artery opening location. In either case, the operator may confirm detection of the renal artery opening by slowly withdrawing the catheter to see if the sensor moves farther away from the catheter in a radial direction. One example, of such movement is shown in FIG. 11. As described above, the position of the sensor can be determined and the determined position used to calculate its distance (e.g., distance X4 between the sensor and the catheter measured from the juncture of the catheter and tapered tip in an orthogonal direction extending radially outward therefrom).

If the aorta was very tortuous, the catheter may have significantly changed the aorta's configuration during advancement therethrough. In this event, the surgeon has the option to take a fluoroscopic image to confirm the location of the renal artery.

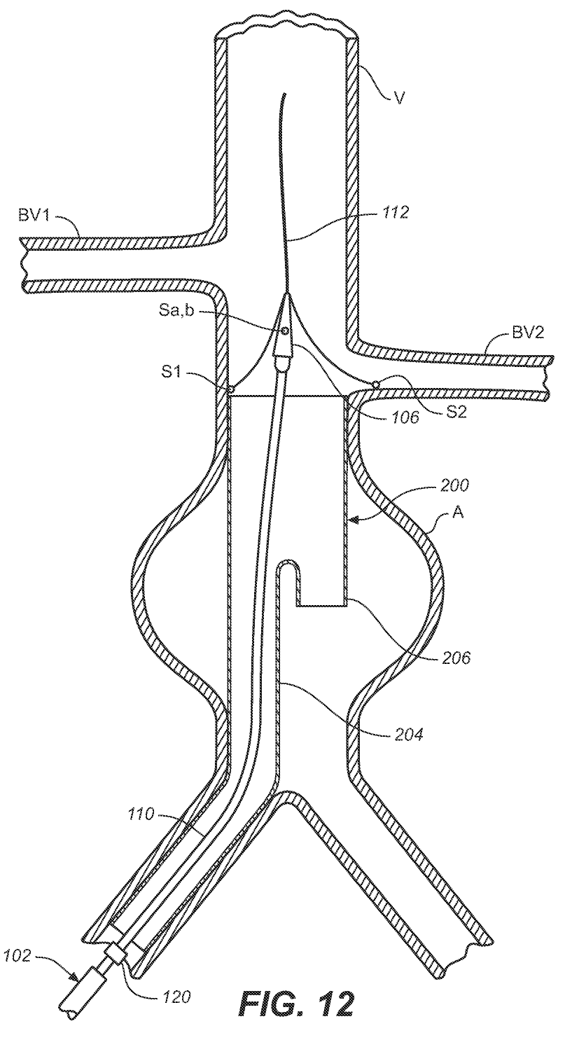

Locating the upper and lower walls of the renal artery provides a guide for the location of the ostium of the renal artery and is related to fiducial markers already present in the anatomy, the stent-graft is positioned at the desired location relative to the three-dimensional model Since the position of the proximal end of stent-graft 200 relative to sensors Sa,b is known, the proximal end of the stent-graft can be positioned at the desired location relative to the renal artery. The catheter is advanced to align sensors Sa,b with S2 while monitoring these sensors on the display and then advances the catheter a distance slightly less than the distance between sensors Sa,b and the stent-graft to align the stent-graft with the proximal neck landing zone. Alternatively, one, two or more sensors can be coupled to the catheter sheath or inner surface of guidewire lumen 110 to indicate the exact position of the proximal end of the stent-graft. Once the stent-graft is in the desired position, the operator holds the guidewire tube 110 and pusher disk 120 stationary and retracts or pulls back sheath 103 (FIG. 12).

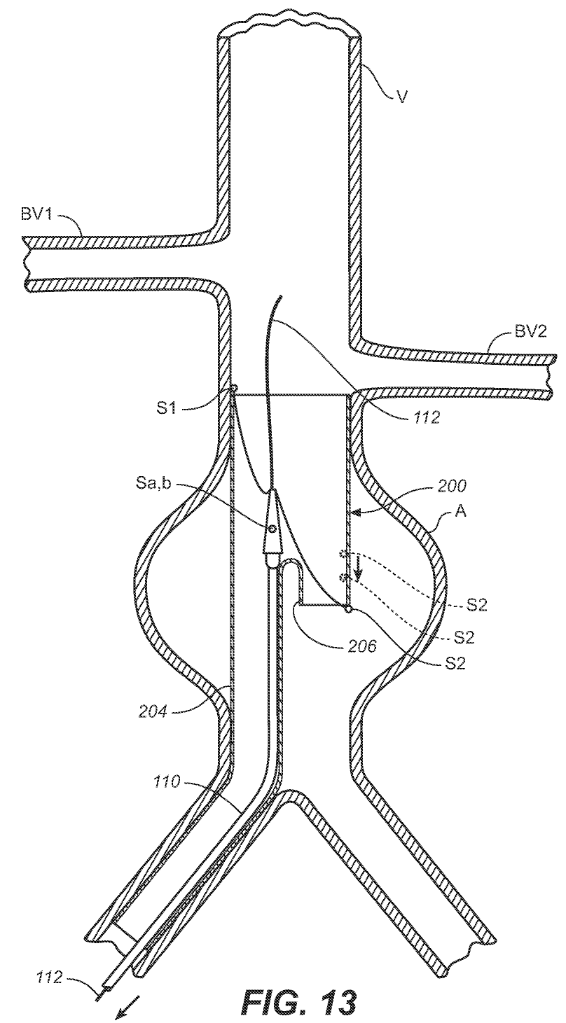

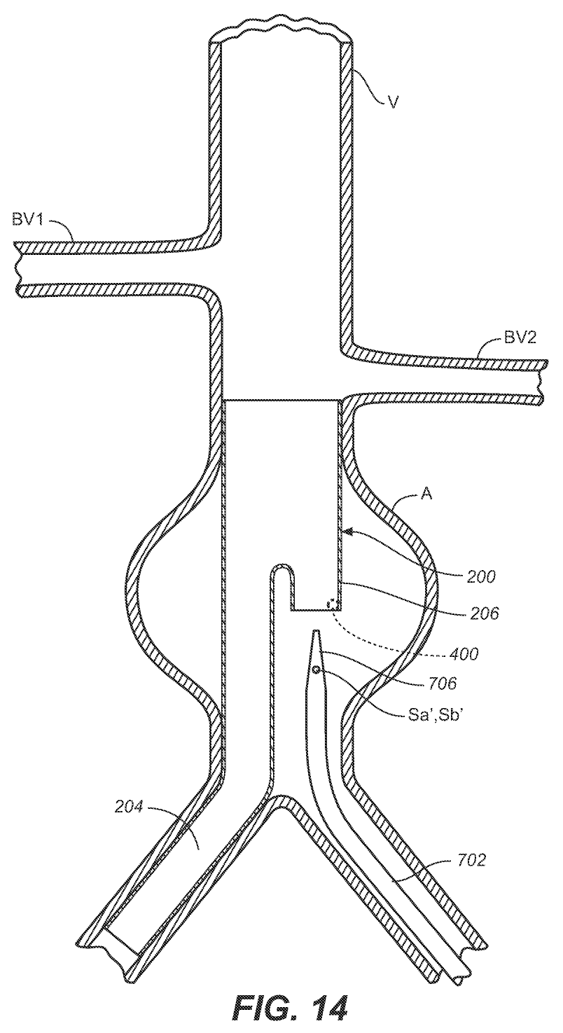

Referring to FIG. 13, the catheter is then retracted to position a sensor (e.g., S2) in the contralateral short leg of modular bifurcated stent-graft 200 as shown in FIG. 13 where the position of S2 is shown in dashed line as it tracks along an inner surface of the short leg until it reaches the end of the short leg from where it moves radially outward. This information allows the operator to record in memory in the three-dimensional image a position inside the contralateral trunk 206 shown in dashed line and designated with reference numeral 400 (FIG. 14).

Referring to FIG. 14, a steerable catheter 702, which can have a similar sheath, guidewire lumen and tapered tip construction as catheter 102, is similarly introduced into the contralateral femoral artery in a conventional manner. Tapered tip 706 includes sensors Sa′ and Sb′, which can be oriented and coupled to tapered tip 706 and constructed in the same manner as sensors Sa and Sb are oriented and coupled to tapered tip 106. As steerable catheter 702 is advanced, the operator uses the three-dimensional model to track tapered tip 706, which leads to the opening of the short leg. If sensor S2 has not been retracted, tapered tip 706 can be guided toward transmitter S2. If sensor S2 has been withdrawn, tapered tip 706 is guided toward position 400. By either moving the sensors closer to one another, while viewing their relative positions as displayed on the monitor or guiding tapered tip 706 toward position 400, while both are displayed on the three-dimensional model, the operator cannulates the contralateral gate of trunk 206 with catheter 702.

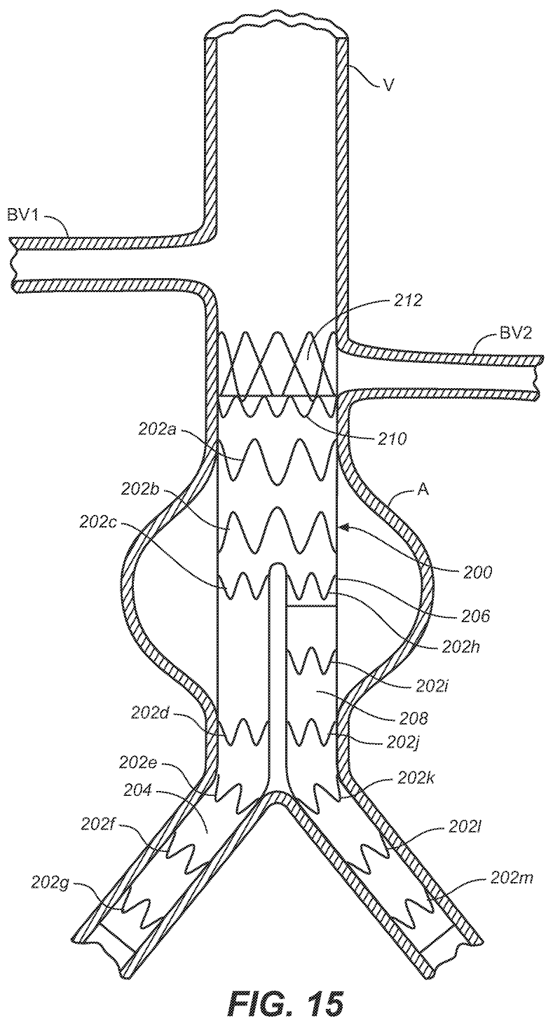

Referring to FIG. 15, the operator then deploys contralateral leg stent-graft section 208 by retracting the catheter sheath in a manner similar to deploying stent-graft 200. The deployed bifurcated stent graft can include a plurality of undulating stents 202a-m secured to the inner or outer wall of the bifurcated tubular graft material (which can comprise, for example, Dacron® or expanded polytetrafluoroethylene (ePTFE)), undulating support wire secured to the inner or outer wall of the proximal portion of the tubular graft, and bare spring 212, which can be secured to the proximal portion of the tubular graft. Bare spring 212 can be flared outwardly moving in a proximal direction to enhance stent-graft anchoring. Sutures or any other suitable means can be used to secure the stents, support wire, and bare spring to the graft material.

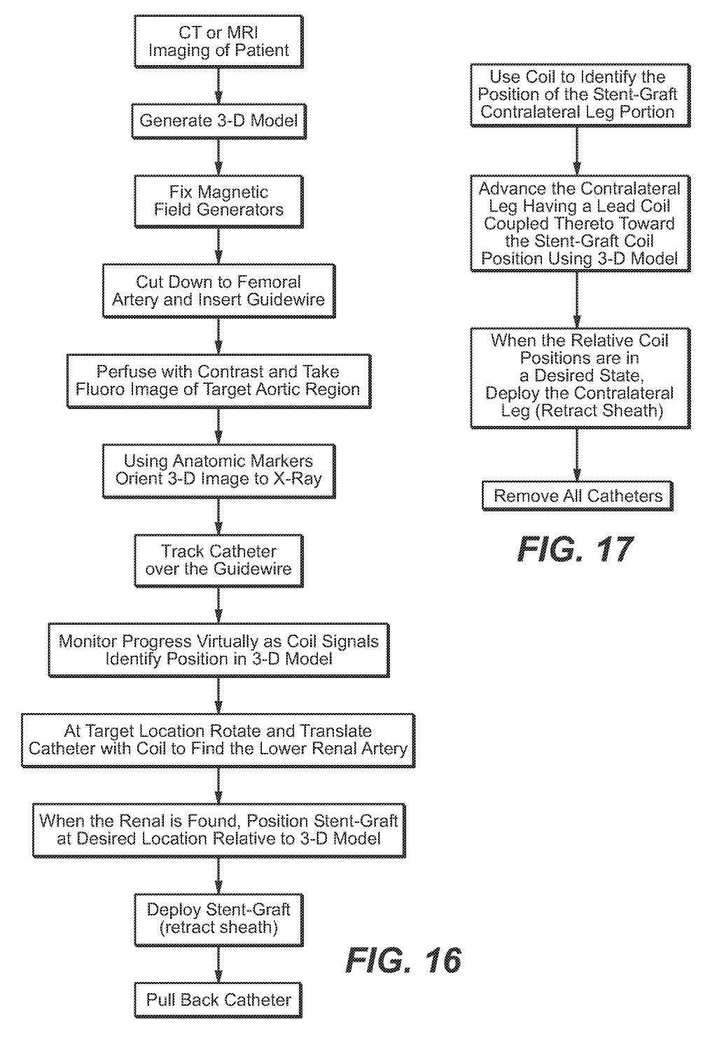

All catheters are then removed. A flow chart summary of the foregoing procedure is depicted in FIGS. 16 and 17.

The three-dimensional data points used in the procedure can increase accuracy of the surgery as compared to two-dimensional fluoroscopic images. The need for contrast agent also can be eliminated or minimized.

In another embodiment according to the invention, a self-contained proximity based system, which does not require external field generators, identifies when the distance between two or more markers or signal devices increases to indicate the position of a branch vessel such as a renal artery.

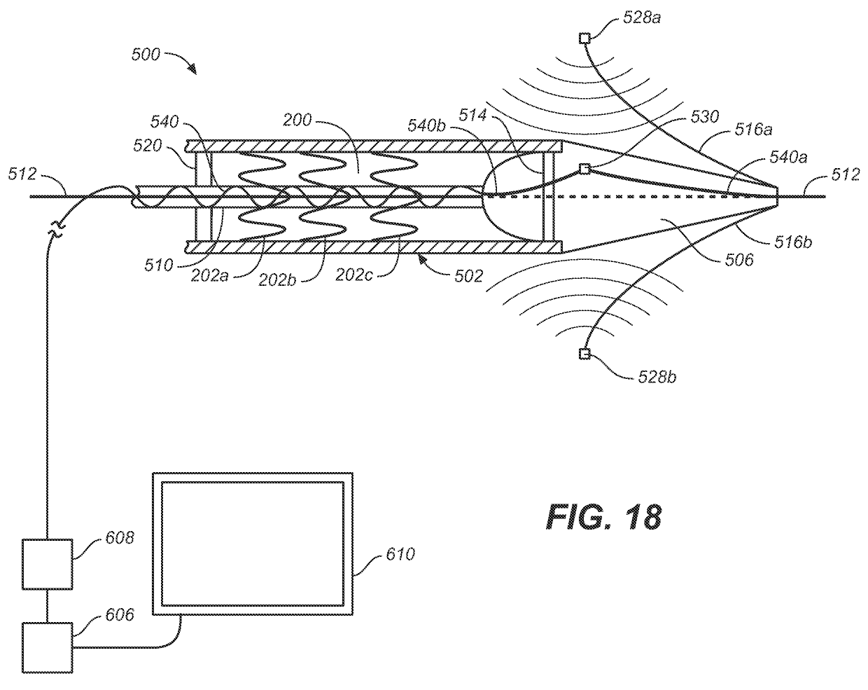

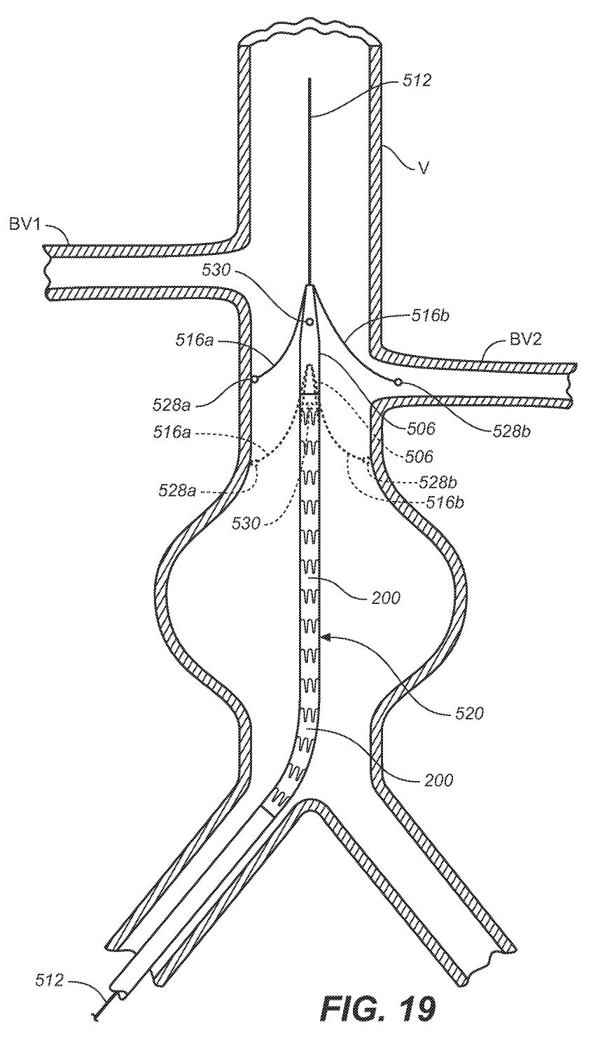

Referring to the illustrative example of FIGS. 18 and 19, stent-graft delivery system 500 includes catheter 502, control handle, tapered tip 506, guidewire lumen 510, guidewire 512, radiopaque ring 514, flexible members 516a and 516b, and pusher disk 520, which can correspond or be similar to catheter 102, control handle, tapered tip, 106 guidewire lumen 110, guidewire 112, radiopaque ring 114, flexible members 116a and 116b, and pusher disk 120.

In this embodiment a signal or wave generating device or transmitter 528a is secured to the feeler end of flexible member 516a or in the vicinity thereof and a signal or wave generating device or transmitter 528b is secured to the feeler end of flexible member 516b or in the vicinity thereof. A conductor can extend from each signal transmitter along a respective flexible member to lead 540a, which extends from the distal end of the tapered tip and then is incorporated into lead bundle 540 where it extends through the guidewire lumen to a power source (not shown) to actuate signal generators 528a and 528b to generate analog RF or infrared electromagnetic signals or waves.

The embodiment illustrated in FIG. 18 also includes a sensor or signal receiver 530, which is embedded or otherwise secured to tapered tip 506 or catheter 502. In an alternative embodiment, receiver 530 can be secured to the distal portion of guidewire lumen 510. Receiver 530 receives the signals from signal generators 528a and 528b and transmits them via lead 540b, which with lead 540a is bundled into lead bundle 540 which is coupled to measuring unit 608, which in turn is coupled to controller 606 and display 610.

Referring to FIG. 18, each signal generator 528a and 528b will be at a fixed distance from sensor 530, the reference position or point, when flexible members 516a and 516b are in a relaxed, undeformed or free state (i.e., in their memory set configuration). As the catheter is advanced through vessel V past aneurysm A as shown in FIG. 19, the flexible members 516a and 516b urge the signal generators against the proximal neck or landing zone of the aneurysm. In this position, the signal generators shown in dashed line. The catheter is further advanced with optional rotation until one signal generator moves into branch vessel BV2 (e.g., a renal artery) to a second position shown in solid line. The movement is in response to the respective flexible member being allowed to move toward its memory shape when it reaches the opening in the vessel wall leading to the branch vessel. The change in the relative position of signal generator 528b and signal receiver 530 versus signal generator 528a and signal receiver 530 indicates that a branch vessel has been detected. The position of 528a to 530, and 528b to 530, and the addition of those two values would be digitally displayed on a monitor.

In a variation of system 500, signal device 530 can be a signal generator and signal devices 528a,b can be signal receivers. As in the embodiment of FIG. 3, a plurality of sensors or sensing coils can be provided on each flexible member in this variation to assist in the proximity evaluation and virtual imaging of the contour, orientation and/or direction of the branch vessel opening. The refinement of the image generally depends on the number of sensors used.

Any of the foregoing embodiments also can be used to obtain three-dimensional data indicative of the opening of branch vessels (e.g. the renal arteries) in applications where there is insufficient proximal neck to anchor the proximal end of the stent-graft. In this case, the stent-graft is positioned across one or both of the branch vessels (e.g., renal arteries) and the acquired position data used to track a steerable piercing catheter having a sensor or signal device coupled to the distal end portion thereof so that the piercing catheter can be guided through the stent-graft and into the either or both branch vessel openings. Alternatively, the stent-graft can include one or more openings, each of which have a recorded position relative to the tapered tip sensor or signal device(s) or one or more sensors attached to the guidewire lumen as described above so that the position of the stent-graft openings can be virtually tracked along the three-dimensional model that has been updated to include the opening position(s).

Any feature described in any one embodiment described herein can be combined with any other feature of any of the other embodiments whether preferred or not.

Variations and modifications of the devices and methods disclosed herein will be readily apparent to persons skilled in the art.

What is claimed is:

1. A method of locating a branch vessel in a human patient comprising;
tracking a sensor moving in a vessel in a first navigational direction;
detecting movement of the sensor in a direction generally orthogonal to the first navigational direction; and
determining if the detected movement is indicative of branch vessel entry.

2. A method of positioning a tubular prosthesis in a passageway in a human body comprising:
advancing a tubular prosthesis through a vessel in a patient;
obtaining the position in three dimensions of a portion of an opening to a branch vessel; and
positioning the proximal end portion of the prosthesis at a predetermined distance from said branch vessel opening portion.

3. The method of claim 2 wherein the vessel is the aorta of the patient and the branch vessel is a renal artery.

4. A method of cannulating a bifurcated tubular prosthesis in vivo comprising:
positioning a bifurcated tubular prosthesis in the aorta of a patient having an ipsilateral leg and a truncated contralateral leg portion;
positioning a first sensor in the truncated contralateral leg portion;
obtaining the position in three dimensions of the first sensor;
advancing a contralateral leg delivery catheter having a distal portion and a proximal portion and a second sensor coupled to the distal portion toward the first sensor position; and
monitoring the second sensor position in three dimensions relative to the first sensor position to guide the distal portion of the contralateral leg delivery catheter into the truncated contralateral leg portion.

5. A prosthesis delivery system comprising:
a tubular prosthesis delivery catheter having a proximal end portion and a distal end portion;
a first sensor coupled to said catheter distal end portion;
a flexible member having a fixed end portion and a feeler end portion, said flexible member fixed end portion being secured to said catheter distal end portion; and
a second sensor coupled to said flexible member feeler end portion and suspended thereby.

6. The delivery system of claim 5 wherein said second sensor is movable relative to said catheter.

7. The delivery system of claim 6 wherein said flexible member comprises a wire.

8. The delivery system of claim 5 wherein said sensors are coils.

9. The delivery system of claim 5 further including a third sensor coupled to said flexible member.

10. The delivery system of claim 9 wherein said second and third sensors are spaced from one another along said flexible member.

11. The delivery system of claim 9 wherein said second and third sensors overlap.

12. The delivery system of claim 9 further including a fourth sensor that is coupled to said flexible member and spaced from said first and third sensors along said flexible member.

13. The delivery system of claim 9 further including a fourth sensor that is secured to said catheter distal end portion.

14. The delivery system of claim 13 wherein said first and fourth sensors overlap.

15. The delivery system of claim 5 wherein said flexible member comprises shape memory material having a first memory set configuration from which it is deformable to and a second configuration from which said flexible member tends to return toward said first configuration.

16. The delivery system of claim 5 further including a prosthesis slidably disposed in said catheter.

17. The delivery system of claim 16 wherein said prosthesis is a stent-graft.

18. The delivery system of claim 5 including a plurality of flexible members and a plurality of sensors secured to said flexible members, each flexible member extending from said catheter distal end portion and having a distal end portion upon which at least one of said sensors is suspended.

19. The delivery system of claim 18 wherein each flexible member comprises shape memory material having a first memory set configuration from which it is deformable to a second configuration from which said flexible member tends to return toward said first configuration

20. The delivery system of claim 5 further including a conductor extending between said catheter proximal end portion and said catheter distal end sensor and a conductor extending between said catheter proximal end portion and said flexible member feeler end portion sensor.

21. The delivery system of claim 5 wherein said catheter comprises a tubular sheath and a tip that is releasably coupled to said tubular sheath and forms at least a portion of said catheter distal end portion, said first sensor being coupled to said tip.

22. A prosthesis delivery system comprising:
a tubular prosthesis delivery sheath having a proximal end portion and a distal end portion;
a tip member having a proximal end portion and a distal end portion, said tip member proximal end portion being releasably coupled to said sheath distal end portion;
a first sensor coupled to said tip member;
a flexible member having a fixed end portion and a feeler end portion, said flexible member fixed end portion being secured to said tip member; and
a second sensor coupled to said flexible member and suspended thereby and being movable relative to said tip member.

23. A stent-graft delivery system comprising:
a stent-graft delivery catheter having a proximal end portion and a distal end portion;
a flexible member having a fixed end portion and a feeler end portion, said flexible member fixed end portion being secured to said catheter distal end portion;
a first sensor coupled to one of said catheter distal end portion and said flexible member;
a signal generator coupled to the other of said catheter distal end portion and said flexible member; and
the one of said sensor and signal generator that is coupled to the flexible member is suspended thereby.

24. A stent-graft delivery system comprising:
a stent-graft delivery sheath having a proximal end portion and a distal end portion;
a tip member having a proximal end portion and a distal end portion, said tip member being releasably coupled to said sheath distal end portion;
a flexible member having a fixed end portion and a feeler end portion, said flexible member fixed end portion being secured to said tip member;
a sensor coupled to one of said tip member and said flexible member;
a signal generator coupled to the other of said tip member and said flexible member; and
the one of said sensor and signal generator that is coupled to said flexible member is suspended thereby and movable relative to said tip member.

25. A probe for locating structure in a patient comprising:
an elongated member configured for endovascular delivery in a patient, said elongated member having a proximal end portion and a distal end portion;
a first sensor coupled to said elongated member distal end portion;
a flexible member having a fixed end portion and a feeler end portion, said flexible member fixed end portion being secured to said elongated member distal end portion; and
a second sensor coupled to said flexible member and suspended thereby.

26. The probe of claim 25 including a plurality of flexible members and a plurality of sensors secured to said flexible members, each flexible member extending from said elongated member distal end portion and having a feeler end portion from which at least one of said sensors is suspended.

27. The probe of claim 26 wherein each flexible member is a wire.

28. The probe of claim 26 wherein each flexible member comprises shape memory material having a first memory set configuration from which it is deformable to a second configuration from which it tends to return toward said first configuration.

29. The probe of claim 26 further including a conductor extending from each of said sensors.

30. The probe of claim 26 wherein each of said sensors is a magnetic field sensing coil.

31. The probe of claim 25 further including a conductor extending from each of said sensors.

32. The probe of claim 25 wherein each of said sensors is a magnetic field sensing coil.
